(19) United States
(12) Patent Application Publication
(10) Pub. No.: US 2002/0183573 A1
(43) Pub. Date: Dec. 5, 2002

(54) Dehydrogenation of an alkyl aromatic compound and catalyst regeneration in a fluidized bed reactor

(76) Inventors: Raymond A Cocco, Midland, MI (US); William M Castor, Lake Jackson, TX (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/130,660

(22) PCT Filed: Nov. 15, 2000
(86) PCT No.: PCT/US00/31305

(51) Int. Cl. C07C 4/06

(52) U.S. Cl. 585/444; 585/445

(57) ABSTRACT

A process of preparing a vinyl aromatic compound, such as styrene. The process involves fluidizing a dehydrogenation catalyst in a single shell fluidized bed reactor containing a reaction zone and a regeneration zone; contacting an alkyl aromatic compound, such as ethylbenzene, with the dehydrogenation catalyst in the dehydrogenation zone so as to produce the vinyl aromatic compound, such as styrene; and regenerating the catalyst in situ by contacting seam with the deactivated catalyst in the regeneration zone. A fluidized bed reactor is described, characterized by a freeboard zone, a reaction zone, and a catalyst regeneration zone, all within a single shell.

This application claims the priority of U.S. Provisional Application Serial No. 60/172,274, filed Dec. 17, 1999.

In one aspect, this invention pertains to a process of dehydrogenating an alkyl aromatic compound, such as ethylbenzene, to form a vinyl aromatic compound, such as styrene. Additionally, the invention pertains to a process of regenerating a catalyst which is used in the dehydrogenation of the alkyl aromatic compound. In another aspect, this invention pertains to a fluidized bed reactor in which an organic process, such as the aforementioned dehydrogenation process, is conducted.

The dehydrogenation of alkyl aromatic compounds, such as ethylbenzene, isopropylbenzene, diethylbenzene, and p-ethyltoluene, find utility in the preparation of styrene and substituted derivatives of styrene, such as α-methylstyrene, divinylbenzene, and p-methylstyrene. Styrene and its substituted derivatives were useful as monomers in the formation of polystyrenes, styrene-butadiene rubbers (SBR), acrylonitrile-butadiene-styrene (ABS), styrene-acrylonitrile (SAN), and unsaturated polyester resins.

Fluidized bed reactors are important in a wide variety of catalyzed organic processes, including dehydrogenation processes.

The chief manufacturing route to vinyl aromatic compounds, such as styrene, is the direct catalytic dehydrogenation of an alkyl aromatic compound, such as ethylbenzene. Patents disclosing such a process include, for example, U.S. Pat. Nos. 4,404,123, 5,171,914, 5,510,552, and 5,679,878. The catalyst typically comprises iron oxide and, additionally, may comprise chromium oxide and potassium compounds, such as potassium hydroxide or potassium carbonate, as promoters. Since the process is highly endothermic, energy for the process is obtained by introducing superheated steam into the process reactor, which in the aforementioned disclosures is typically a fixed bed design. Process temperatures are generally between about 550° C. and 700° C. Side reactions can be controlled by maintaining a low partial pressure of ethylbenzene.

There are several disadvantages in using a fixed bed reactor for the above-described dehydrogenation processes. First, a fixed bed reactor, characterized by a stationary bed of catalyst particles, is difficult to heat uniformly to a high temperature. In the endothermic dehydrogenation of ethylbenzene to styrene, the downstream (exit) end of the fixed catalyst bed tends to be colder than the upstream (entrance) end of the catalyst bed. Since the temperature differential may reduce the ethylbenzene conversion at the downstream end of the reactor, the feedstream is typically preheated to a temperature higher than desirable. As a consequence, the catalyst bed near the entrance to the reactor may be overheated and typically deteriorates faster than the catalyst farther downstream. Under the circumstances, a large catalyst bed is needed to sustain a long running cycle. On another point, in fixed bed processes for dehydrogenating ethylbenzene to styrene, steam is fed concurrently with the ethylbenzene to promote catalyst regeneration in situ during the dehydrogenation process. Usually, a high steam to ethylbenzene weight ratio is required, typically from greater than 1.2 to 2.0 and possibly higher, which disadvantageously imposes on the process a high energy input and a large water recycle. (The steam to alkyl aromatic compound weight ratio is hereinafter referred to as the “steam to oil ratio.”) When the overall bed activity diminishes beyond the point of practicality, the catalyst must be replaced. As a further disadvantage, fixed bed reactors are typically shut down for weeks for catalyst replacement.

Other references, such as U.S. Pat. Nos. 3,651,146 and 4,471,146, disclose oxidative dehydrogenation processes wherein ethylbenzene is contacted with oxygen in a fluidized bed reactor in the presence of an oxidative dehydrogenation catalyst, for example, an alkaline earth-nickel phosphate or an alkali metal-chromium oxide composite, to produce styrene. A conventional fluidized bed reactor comprises a single reaction zone wherein catalyst particles are disengaged and circulating. As compared with fixed beds, fluidized bed reactors provide a more isothermal temperature distribution. An isothermal catalyst bed is typically less damaging to the catalyst and allows for better product yields. Fluidized beds also allow for easy replacement of the catalyst when it is fully deactivated and incapable of regeneration. Since the catalyst is treated as a fluid, deactivated catalyst can be discharged from the reactor and active catalyst can be added to the reactor without shutting down the chemical process. The aforementioned oxidative dehydrogenation process may also be conducted while continuously transporting a portion of the catalyst to a regenerator for regeneration under oxygen, and then recirculating the regenerated catalyst back to the oxidative dehydrogenation reactor. On the down side, oxidative dehydrogenation processes with co-feeds of alkyl aromatic compound and oxygen may produce low yields of vinyl aromatic product, because oxidation side reactions are more difficult to control. Additionally, safety issues involved with handling and processing mixtures of organic compounds and oxygen are significant. As a further disadvantage, the continuous cycling of catalyst between the fluidized bed process reactor and the regenerator requires complex equipment and often requires a highly attrition resistant catalyst particle.

Some patents, such as U.S. Pat. Nos. 4,229,604 and 5,510,553, disclose the use of a transport reactor for dehydrogenating ethylbenzene to styrene in the absence of oxygen and in the presence of an oxidative dehydrogenation catalyst, such as, silica modified with magnesia or a reducible oxide of vanadium supported on a metal oxide. These processes eliminate the hazard of employing mixtures of alkyl aromatic compound and oxygen; however, the lifetime of such catalysts is brief. Accordingly, the catalyst must be circulated continuously between the process reactor and a regenerator wherein the catalyst is regenerated under oxygen. As noted hereinbefore, the continuous recycling of catalyst between the process reactor and the regenerator requires complex equipment and often requires a highly attrition resistant catalyst particle.

The aforementioned description suggests a need for improving catalytic dehydrogenation processes. It would be advantageous, for example, to discover a process for dehydrogenating an alkyl aromatic compound, such as ethylbenzene, to a vinyl aromatic compound, such as styrene, which provides the efficient in situ regeneration of the catalyst at an economical steam to oil ratio. It would be advantageous if during the process, deactivated catalyst could be replaced with active catalyst without the necessity of shutting down the reactor or using complex transport equipment. It would be advantageous, if an isothermal bed temperature could be maintained. It would also be advantageous, if the process did not require oxygen which complicates safety and handling procedures. Finally, the process would be even more desirable if it possessed all of the aforementioned features and also achieved a high yield of vinyl aromatic compound.

In another aspect, U.S. Pat. No. 4,152,393 discloses a reactor consisting of a single shell that contains a reaction zone and a regeneration zone arranged in such a manner, specifically, as a collection of concentric walls and paths, that a particulate solid may be transferred by flow of gases from the regeneration zone to the reaction zone by a first route and then back to the regeneration zone by a second route. The gases passing through the regeneration zone are not transferred to the reaction zone, and the gases passing through the reaction zone are not transferred to the regeneration zone. It is taught that this reactor is useful for the ammoxidation of propylene. The disclosed reactor may exhibit high slug flow, characterized by gas bubbles flowing along the inner walls of the reactor. Slug flow disadvantageously decreases contact between the gas phase reactants and the solid catalyst particles, thereby decreasing the productivity of the process. As a further disadvantage, this reactor possesses narrow curved spaces and numerous gas jets within those spaces, which may lead to a high attrition of catalyst particle. U.S. Pat. No. 6,048,459 discloses a method of fluidization of a particulate bed material involving collecting and uplifting a portion of a fluid above a fluidized bed and recycling the lifted fluid under the bed via a downcomer positioned within the bed. A zone for particulate material may be extended under the fluidized bed for use, specifically, in treating anaerobic sludge over a long time.

In one aspect, this invention is a process of dehydrogenating an alkyl aromatic compound over a dehydrogenation catalyst in a single shell, fluidized bed reactor to form a vinyl aromatic compound, and regenerating in situ the dehydrogenation catalyst. The process of this invention comprises, in a first step, (a) fluidizing a dehydrogenation catalyst in a single shell, fluidized bed reactor containing a reaction zone and a regeneration zone under fluidization conditions such that the catalyst is circulated within and between the two zones; (b) contacting an alkyl aromatic compound, and optionally, steam with the dehydrogenation catalyst residing in the reaction zone under reaction conditions sufficient to prepare the corresponding vinyl aromatic compound; and (c) contacting steam with the dehydrogenation catalyst residing in the regeneration zone, the contacting being conducted under regeneration conditions sufficient to regenerate, at least in part, the catalyst.

The dehydrogenation process of this invention, which finds utility in the preparation of vinyl aromatic compounds of industrial significance, such as styrene, pmethylstyrene, α-methylstyrene, and divinylbenzene, possesses significant advantages over prior art processes. First, the process of this invention does not employ oxygen. Accordingly, safety problems associated with handling and processing mixtures of organic compounds and oxygen, which are found in some prior art processes, are eliminated in the process of this invention. Secondly, in the process of this invention, the steam to oil ratio is advantageously lower than that used in prior art fixed bed processes. Accordingly, the process of this invention uses a smaller water recycle and is more energy efficient and economical than prior art processes. As an added advantage, the process of this invention, being conducted in a fluidized bed reactor, is essentially isothermal. Problems associated with non-uniform bed temperatures, such as, overheating and catalyst damage at the upstream end of the catalyst bed and lowered productivity at the downstream end of the catalyst bed, are essentially eliminated. Also, thermal byproduct formation is reduced. As a further advantage, a smaller catalyst bed may be used in the process of this invention, as compared with the size of the catalyst bed used in fixed bed prior art processes, while still achieving comparable running cycles. As another advantage, the process of this invention provides for the continuous in situ regeneration of the dehydrogenation catalyst. There is no necessity in the process of this invention to shut the reactor down for catalyst regeneration or to transport the catalyst out of the fluidized bed reactor to a regenerator. Accordingly, the process of this invention possesses a simplicity of design and operation. Moreover, the catalyst used in the process of this invention may not require the high attrition resistance needed for transport reactors. Finally, when the catalyst is incapable of further regeneration, the process of this invention provides for the replacement of deactivated catalyst during continuous operation of the dehydrogenation process. Since the solid catalyst is treated as a fluid, the deactivated catalyst is simply conveyed out of the reactor, and fresh catalyst is conveyed into the reactor, during operation. Thus, regeneration and replacement of the catalyst can both be accomplished without shutting down the dehydrogenation process, thereby resulting in higher productivity. Most advantageously, the process of this invention produces vinyl aromatic compound, preferably styrene, in high yield.

In another aspect, this invention is a fluidized bed reactor which allows for chemical processing and catalyst regeneration simultaneously. The fluidized bed reactor of this invention comprises a single, vertical shell within which the space is divided into a freeboard zone, a reaction zone, and a regeneration zone. The reactor also comprises an inlet means for introducing a regeneration feedstream into the regeneration zone, and an inlet means for introducing a reactant feedstream into the reaction zone. The reactor further comprises a means for separating the reaction and regeneration zones while allowing for the continuous circulation and large scale backmixing of the catalyst between the two zones. In a preferred embodiment, one of the inlet means for the reaction or regeneration feed acts as the means for separating the reaction and regeneration zones. The reactor of this invention also comprises an outlet means, preferably in the freeboard zone, for removing an effluent stream containing products and any unconverted reactants and regeneration feeds. Optionally, the reactor further contains a means for returning catalyst particles entrained in the effluent stream back to the reactor. Optionally, also, an inlet means and an outlet means may be present for conveying catalyst into and out of the reactor.

In the reactor of this invention, cross-mixing of the reactant and regeneration feedstreams may occur; although, preferably, the placement of the reaction and regeneration inlets will substantially separate both processes. Nevertheless, with backmixing of gases and solids being allowed in this design, the regeneration and reactant feedstreams should be chemically compatible, as illustrated herein with the instant dehydrogenation-regeneration process.

The fluidized bed reactor of this invention can be used in a variety of catalyzed organic processes, including, for example, dehydrogenations, oxidations, and halogenations. A particularly important dehydrogenation process, for which the fluidized bed reactor of this invention can be used, comprises the dehydrogenation of an alkyl aromatic compound, such as ethylbenzene, to a vinyl aromatic compound, such as styrene. Beneficially, the fluidized bed reactor of this invention provides a chemical process reaction zone and a catalyst regeneration zone within a single fluidized bed reactor shell. Accordingly, the in situ regeneration of the catalyst can be achieved simultaneously with the desired chemical process. The deactivated catalyst need not be transported out of the fluidized bed reactor of this invention to a separate vessel for regeneration; thus, the catalyst is subjected to far less stress and damage than that experienced in transport reactors. As another advantage, deactivated catalyst can be replaced on-line without shutting down the chemical process, by simply conveying deactivated catalyst out of the reactor and conveying fresh catalyst into the reactor. These advantages provide needed improvements in fluidized bed process engineering.

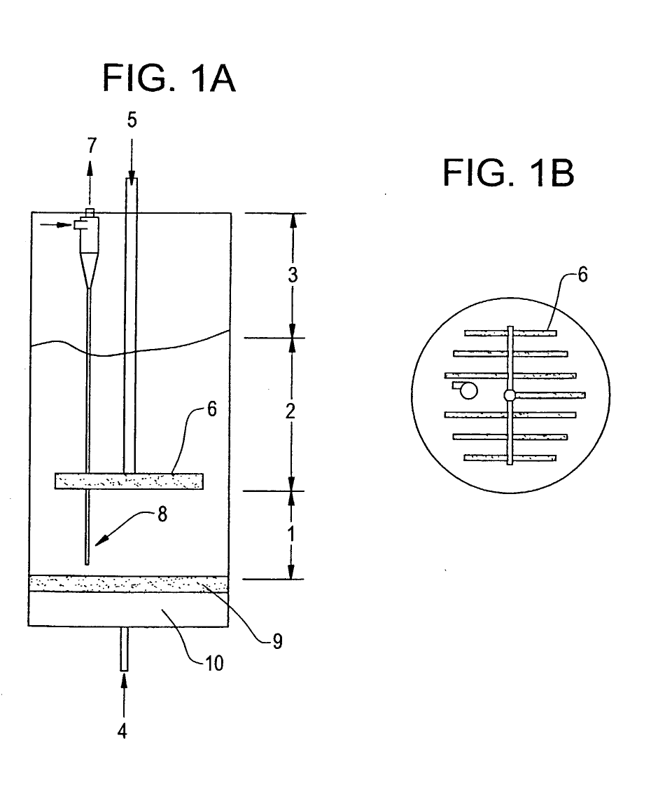

FIG. 1 shows cross-sectional side and top views of a first preferred embodiment of the fluidized bed reactor of this invention, details of which are set forth hereinafter.

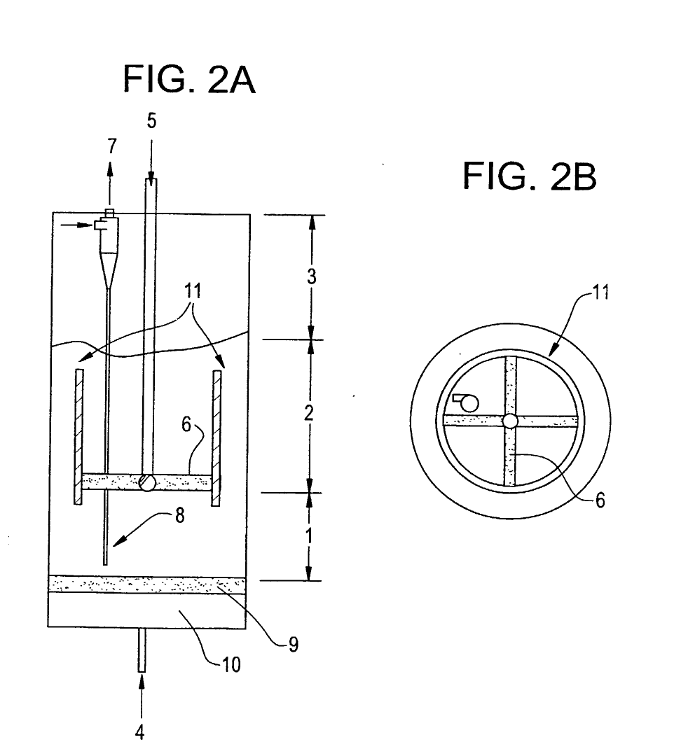

FIG. 2 shows cross-sectional side and top views of a second preferred embodiment of the fluidized bed reactor of this invention, details of which are set forth hereinafter.

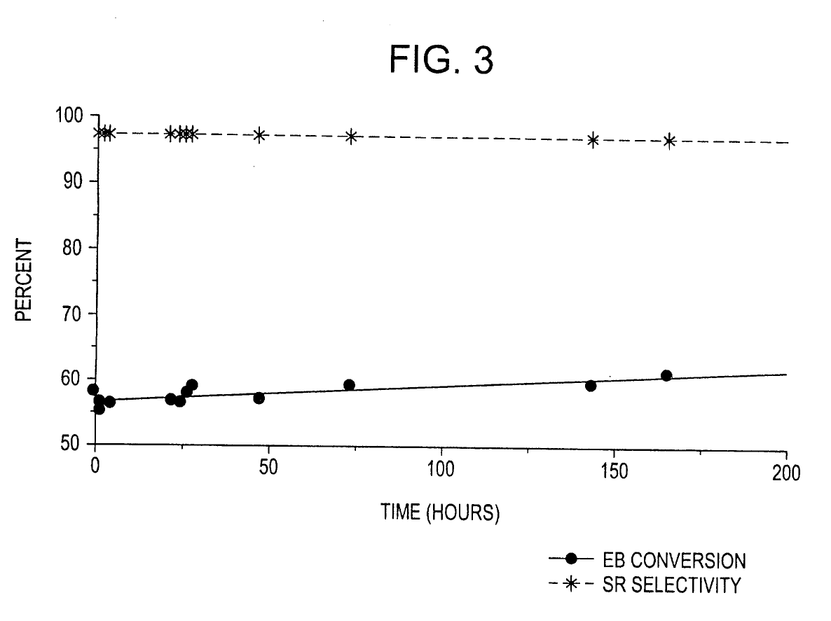

FIG. 3 is a plot of ethylbenzene conversion and styrene selectivity as a function of run time for an ethylbenzene dehydrogenation and catalyst regeneration process conducted in a pulsed mode reactor.

In one aspect, this invention is a process of dehydrogenating an alkyl aromatic compound over a dehydrogenation catalyst in a single shell, fluidized bed reactor to form a vinyl aromatic compound, and regenerating in situ the dehydrogenation catalyst. The process of this invention comprises (a) fluidizing a dehydrogenation catalyst in a single shell, fluidized bed reactor containing a reaction zone and a regeneration zone under fluidization conditions such that the catalyst is circulated within and between the two zones. In a second step which is conducted simultaneously with the first step, the process comprises (b) contacting an alkyl aromatic compound, and optionally, steam with the dehydrogenation catalyst residing in the reaction zone under reaction conditions sufficient to prepare the corresponding vinyl aromatic compound. In a third step, which is conducted preferably simultaneously with the first and second steps, the process comprises (c) contacting steam with the dehydrogenation catalyst residing in the regeneration zone, the contacting being conducted under regeneration conditions sufficient to regenerate, at least in part, the catalyst.

In the fluidized bed process of this invention, at any given instant in time a portion of the catalyst will be circulating in the reaction zone, while essentially the remainder of the catalyst will be circulating in the regeneration zone, with some co-mixing at the boundary of the two zones. Over a period of time catalyst residing in the reaction zone will lose activity and become partially or fully deactivated. Deactivation may be mostly caused by a build-up of coke on the surface of the catalyst. (The invention should not be bound or limited, however, by such a deactivation theory.) Under fluidization conditions, catalyst in the reaction zone, including deactivated catalyst, will circulate into the regeneration zone. Deactivated catalyst residing in the regeneration zone will be reactivated by contact with steam. Reactivation results in partial or essentially full recovery of catalyst activity, as compared with the activity of the fresh (unused or “as-synthesized”) catalyst. Thereafter under the fluidization conditions, the regenerated catalyst in the regeneration zone will be recycled to the reaction zone, and the reaction/-regeneration cycle will be repeated again and again. The aforementioned description is given as a means of explaining the reaction-regeneration cycle and of defining the words “to regenerate, at least in part, the catalyst.”

It will also be understood that after repeated reaction and regeneration, there comes a time when the catalyst can no longer be regenerated to a practical level of activity, even with the regeneration procedure described herein. When this occurs, the deactivated catalyst can be replaced simply by conveying it out of the reactor and conveying fresh catalyst into the reactor, preferably simultaneously. In the reactor of this invention, catalyst replacement can be conducted “on-line” without shutting down the catalytic process, more particularly to this invention, the dehydrogenation process. The catalyst can be conveyed into and out of the reactor via air jets or a pneumatic transport loop. Alternatively, catalyst can be removed from the reactor via a gravity-driven outlet means at the bottom of the reactor, and catalyst can be added to the reactor from a standpipe inlet means at the top of the reactor.

In a preferred aspect of this invention, the alkyl aromatic compound is ethylbenzene or a substituted derivative of ethylbenzene, and the vinyl aromatic compound produced is styrene or a substituted derivative of styrene.

Any alkyl aromatic compound can be employed in the dehydrogenation process of this invention, provided that the product achieved is a vinyl aromatic compound. The aromatic moiety of the vinyl aromatic compound can comprise, for example, a monocyclic aromatic ring, such as phenyl; a fused aromatic ring, such as naphthyl; or a ring assembly, such as biphenylyl. Preferably, the aromatic moiety is a monocyclic aromatic ring, more preferably, phenyl. The alkyl moiety of the alkyl aromatic compound can comprise, for example, any saturated straight chain, branched, or cyclic hydrocarbon radical, provided that it can be dehydrogenated in the process of this invention to a vinyl moiety. Non-limiting examples of suitable alkyl moieties include ethyl, n-propyl, iso-propyl, nbutyl, iso-butyl, t-butyl, and higher homologues thereof. Preferably, the alkyl moiety is a C₂-C₁₀ alkyl, more preferably, a C₂-C₅ alkyl, most preferably, ethyl. The alkyl aromatic compound may be substituted optionally with two or more alkyl moieties, or substituted with other types of substituents which are essentially inert with respect to the dehydrogenation process of this invention. Examples of alkyl aromatic compounds which are beneficially employed in the process of this invention include, without limitation, ethylbenzene, diethylbenzene, ethyltoluene, ethylxylene, isopropylbenzene, t-butylethylbenzene, ethylnaphthalene, ethylbiphenyl, and higher alkylated homologues thereof Preferably, the alkyl aromatic compound is a C₈-C₂₀ alkyl aromatic compound, more preferably, a C₈-C₁₅ alkyl aromatic compound, and most preferably, ethylbenzene or a substituted derivative thereof.

In the process of this invention, the regeneration feedstream typically comprises steam. Optionally, steam may also be incorporated into the dehydrogenation feedstream. Any weight ratio of steam to alkyl aromatic compound (steam to oil ratio) is suitable for the process of this invention, provided that the process produces a vinyl aromatic compound. Note that the steam to oil ratio is based on the total weight of the steam introduced into the reactor from all sources, including steam from both the dehydrogenation and the regeneration feedstreams. Typically, the steam to oil weight ratio is greater than about 0.2/1, preferably, greater than about 0.5/1. Typically, the steam to oil weight ratio is less than about 5.0/1, preferably, less than about 3.0/1, even more preferably, less than about 1.2/1, and most preferably, less than about 1.0/1. Generally, the process of this invention operates at lower steam to oil ratios, as compared with prior art processes. A low steam to oil ratio advantageously reduces the energy requirement and cost of converting water to steam and reduces the quantity of water recycled to the reactor.

Optionally, a sweeping gas may be used in the process of this invention. The sweeping gas, which may be introduced directly into the freeboard zone of the reactor, primarily functions to remove the product stream from the freeboard zone, where undesirable thermal reactions can occur. Any gas which is substantially inert with respect to the dehydrogenation and regeneration processes may be suitably employed as the sweeping gas, including, for example, nitrogen, argon, helium, carbon dioxide, steam, and mixtures thereof. The concentration of sweeping gas in the freeboard zone can be any concentration, provided that the overall process produces the desired vinyl aromatic compound. Generally, the concentration of sweeping gas varies depending, for example, upon the specific alkyl aromatic compound and specific process conditions employed, particularly, the temperature and gas velocity. Typically, the concentration of sweeping gas in the freeboard zone is greater than about 10 volume percent, and preferably, greater than about 20 volume percent. Typically, the concentration of sweeping gas in the freeboard zone is less than about 90 volume percent, and preferably, less than about 70 volume percent.

Optionally, the dehydrogenation and/or regeneration feedstream(s) may also contain a diluent. The diluent primarily dilutes the reactants and products for improved selectivity or safety considerations. Any gas which is substantially inert with respect to the dehydrogenation and regeneration steps may be suitably employed as the diluent, including, for example, nitrogen, argon, helium, carbon dioxide, steam, and mixtures thereof. The concentration of diluent in either the dehydrogenation or regeneration feedstream can be any concentration, provided that the overall process produces the desired vinyl aromatic compound. Generally, the concentration of diluent varies depending, for example, upon the specific diluent chosen, the specific alkyl aromatic compound, the specific dehydrogenation or regeneration process conditions, and the specific catalyst and its deactivation properties. Typically, the concentration of diluent in the dehydrogenation or the regeneration feedstream is greater than about 10 volume percent, preferably, greater than about 20 volume percent. Typically, the concentration of diluent in either stream is less than about 90 volume percent, preferably, less than about 70 volume percent. When steam is employed as the diluent, then the steam to oil weight ratio, described hereinabove, determines the concentration of steam in the dehydrogenation feedstream.

Oxygen is not required for the process of this invention. Preferably, oxygen is not employed in the process of this invention.

Any dehydrogenation catalyst which is capable of catalyzing the dehydrogenation of an alkyl aromatic compound to a vinyl aromatic compound can be used in the process of this invention. Non-limiting examples of dehydrogenation catalysts which can be beneficially employed include the catalysts described in the following U.S. patents: U.S. Pat. Nos. 4,404,123, 4,503,163, 4,684,619, 5,171,914, 5,376,613, 5,510,552, and 5,679,878, which pertain to a variety of iron oxide catalysts, containing, for example, one or more compounds of the alkali metals, preferably, sodium, potassium, and cesium; alkaline earth metals, preferably, calcium; and/or cerium, chromium, zinc, copper, and/or gallium compounds, as well as, the catalyst described in U.S. Pat. No. 3,651,160, which pertains to chromium oxide and alkali metal oxides. Preferably, the catalyst is a dehydrogenation catalyst comprising iron oxide. More preferably, the catalyst comprises (a) at least one iron oxide, (b) at least one carbonate, bicarbonate, oxide or hydroxide of potassium and/or cesium, (c) an oxide, carbonate, nitrate or hydroxide of cerium, (d) optionally, a hydroxide, carbonate, bicarbonate, acetate, oxalate, nitrate, or sulfate of sodium, (e) optionally, a carbonate, sulfate, or hydroxide of calcium, and (f) optionally, one or more binding agents, such as, a hydraulic cement. As a further option, the more preferred catalyst may additionally comprise one or more oxides selected from zinc, chromium, and copper. Typically, the more preferred catalyst comprises from 25 to 60 weight percent iron, from 13 to 48 weight percent potassium, and from 1 to 20 weight percent cerium, the weight percentages being calculated as the oxides. These proportions and other proportions of suitable catalytic components are described in the aforementioned US patents.

The dehydrogenation catalyst which is used in the fluidized bed reactor of this invention can possess any particle size or shape, so long as the catalyst is capable of catalyzing the dehydrogenation of an alkyl aromatic compound to a vinyl aromatic compound. Typically, the average catalyst particle size is greater than about 20 microns(μm) in diameter (or cross-sectional dimension), and preferably, greater than about 50 μm in diameter. Typically, the average particle size is less than about 1,000 μm, and preferably, less than about 200 μm. Preferably, the catalyst particle is smooth with rounded edges, is substantially non-cohesive, and possesses an attrition resistance sufficient for use in a fluidized bed reactor. One skilled in the art will know whether a particular catalyst has sufficient attrition resistance for use in a fluidized bed reactor.

If desired, the dehydrogenation feedstream may be preheated prior to its introduction into the reaction zone. The preheat can be conveniently supplied by condensing high pressure saturated steam, or alternatively, by combusting a fuel source or process off-gas. Any preheat temperature can be used, provided it lies below the temperature at which thermal cracking of the alkyl aromatic compound becomes measurable. Typical preheat temperatures are greater than about 150° C., preferably, greater than about 250° C., and more preferably, greater than about 350° C. Typical preheat temperatures are less than about 600° C., and preferably, less than about 590° C. Likewise, the regeneration feedstream can be preheated prior to its introduction into the regeneration zone. A typical preheat temperature for the regeneration stream is greater than about 200° C., preferably, greater than about 300° C., and more preferably, greater than about 400° C. The preheat temperature of the regeneration feedstream is typically less than about 650° C., and preferably, less than about 630° C.

A preferred embodiment of the novel reactor which is employed in the process of this invention is the fluidized bed reactor shown in FIG. 1, comprising a single vertical shell whose inner space is functionally divided into a regeneration zone (1), a reaction zone (2), and a freeboard zone (3). The regeneration zone, located in this preferred embodiment at the bottom of the reactor, comprises the region wherein catalyst is regenerated. The reaction zone, situated in this preferred embodiment in the middle zone of the reactor, is the region wherein the catalyzed organic chemical process occurs, such as the dehydrogenation process described herein. The freeboard zone, located at the top of the reactor, comprises the space above the middle zone up to the top inner wall of the reactor.

The freeboard zone, occupied by gaseous reactants and products, also provides space for expansion of the fluidized bed. Gas phase thermal reactions may occur in the freeboard zone; but process conditions are preferably maintained which minimize these gas phase reactions relative to the catalyzed process occurring in the reaction zone.

The regeneration zone of FIG. 1 contains an inlet means for introducing the regeneration feedstream, herein steam and, optionally, a diluent into the regeneration zone.

The inlet means can comprise, for example, a inlet port (4), which exits into a plenum (10) above which a distributor plate or sparger array (9) is situated. The freeboard zone contains an inlet means (5) comprising, for example, an inlet port and delivery tube, for introducing the reactant feedstream, herein the dehydrogenation feedstream, into the reaction zone. In FIGS. 1 and 2, the inlet means for the regeneration zone is shown at the bottom of the figure, and the inlet means for the reaction zone is shown at the top of the figure. In practice, the regeneration zone inlet means can be situated anywhere, provided that it exits in the regeneration zone. Likewise, the reaction zone inlet means can be situated anywhere, so long as the reactants are fed into the reaction zone.

Preferably, the inlet means (5) terminates in a sparger array or distributor (6) in the reaction zone, located more preferably, at a level above the bottom steam distributor (9). The distributor or sparger array (6) is preferably designed to deliver the reaction feedstream in any direction into the reaction zone. The distance from the bottom steam distributor (9) to the reactant feedstream distributor (6) can be varied to provide for variable volumes in the regeneration and reaction zones, as desired. The larger the zone, the longer will be the residence time of gases and solids in that zone. As described herein, the distributor means (6) provides a functional demarcation between the regeneration zone and the reaction zone, such that the regeneration process substantially occurs in the regeneration zone while the catalyzed organic process substantially occurs in the reaction zone, while still allowing for backmixing of solids and gases. The gas distributors and sparger arrays can be manufactured, for example, from gas permeable sintered metal, or more preferably, the gas distributors and spargers can be fitted with jets for dispersing the gas. The freeboard zone also contains an outlet means (7), such as an outlet port, for the effluent stream, comprising unconverted alkyl aromatic compound, steam, the optional sweeping gas and/or diluent, and products, including the vinyl aromatic compound. The outlet means (7) may be connected to a cyclone (shown in FIG. 1, below outlet 7) for collecting catalyst particles which are entrained with the effluent stream. The collected catalyst particles can be recycled to the fluidized bed reactor via inlet means (8), situated at any point along the reactor, but preferably, as shown in FIG. 1 into the regeneration zone. The outlet means (7) is further connected to a separations unit (not shown in FIG. 1), including for example, a condensation means and a distillation train for separating unconverted alkyl aromatic compound and reaction products. Unconverted reactants may be recycled back to the reaction zone via inlet (5). In addition to the above, the reactor may further comprise a means for measuring the temperature of the catalyst bed and, optionally, a means for heating the reactor (not shown in figures). The reaction zone and regeneration zones may also contain baffles (not shown in figures) which function to reduce the formation and size of bubbles, thereby facilitating contact between the gaseous feedstreams and the catalyst.

In another preferred embodiment of this invention, the fluidized bed reactor additionally contains one or more means for enhancing solids circulation and heat transfer.

In a preferred embodiment, the means for enhancing solids circulation comprises one or more draft tubes, optionally, containing internal baffles. Alternatively, the means for enhancing solids circulation comprises one or more draft tubes made of heating or cooling elements. An embodiment of the invention containing a plurality of draft tubes is shown in FIG. 2. (Parts 1-10 of FIG. 2 are identical to parts 1-10 of FIG. 1.) The draft tubes (11) may each comprise, for example, concentric cylinders open at both ends, or a bundle or array of heating tubes, or any other design which promotes drafting of the catalyst. Typically, the draft tube is vertically suspended through the reaction and regeneration zones, preferably, to near the top of the reaction zone. The dehydrogenation feedstream is fed through the inlet port (5) into the sparger array (6) and up the inner cylinder of the draft tube (11) into the reaction zone. As a consequence of the fluidization conditions, catalyst particles will become entrained in the inner cylinder of the draft tube and transported up to the top of the draft tube. At the top, the catalyst particles will flow over the lip of the inner cylinder and down through the annular region between the two cylinders back into the regeneration zone.

In addition to the above, the reactor may optionally comprise an inlet means and an outlet means (not shown in the figures) for respectively transporting catalyst into and out of the reactor.

In another embodiment of the fluidized bed reactor, the reaction and regeneration zones can be reversed, such that the reaction zone is located at the bottom of the reactor while the regeneration zone is located in the middle of the reactor. (FIG. 1 wherein the reaction zone is located at (1), the regeneration zone is located at (2), and the inlets thereto are adjusted accordingly.)

The reactor of this invention can be employed in catalytic processes wherein the reactant feedstream is chemically compatible with the regeneration feedstream. The unique reactor of this invention allows for the continuous flow of catalyst particles between reaction and regeneration zones within a single shell fluidized bed reactor. Accordingly, the catalyzed organic process of interest and the catalyst regeneration can be effected simultaneously without transporting catalyst out of the reactor into a separate regenerator. The reactor of this invention does not contain complex concentric walls or winding paths through which the catalyst particles traverse. Accordingly, for large scale units, the reactor of this invention does not produce significant slug flow and attrition problems.

The temperature of the reaction zone wherein the dehydrogenation process occurs can be any operable temperature, provided that a vinyl aromatic compound is produced in the process. The operable dehydrogenation temperature will vary with the specific catalyst and specific alkyl aromatic compound employed. For the preferred catalyst containing iron oxide, the dehydrogenation temperature is typically greater than about 550° C., and preferably, greater than about 570° C. Typically, the dehydrogenation temperature is less than about 650° C. and, preferably, less than about 610° C. Below about 550° C., the conversion of alkyl aromatic compound may be too low; whereas above about 650° C., thermal cracking of the alkyl aromatic compound and vinyl aromatic product may occur. In this invention, temperature is measured on the catalyst bed in fluidized form.

In the regeneration zone, the catalyst is contacted with steam and reactivated. The temperature of the regeneration zone can also be varied, so long as the catalyst is at least partially regenerated. Typically, the regeneration temperature lies below the thermal cracking temperature for the alkyl aromatic reactant and vinyl aromatic product. For the preferred catalyst containing iron oxide, the regeneration temperature is typically greater than about 550° C., and preferably, greater than about 570° C. Typically, the regeneration temperature is less than about 650° C. and, preferably, less than about 610° C. Since the catalyst is recirculating continuously between the reaction and regeneration zones and since the temperatures of the two zones are maintained at closely similar values, the fluidized bed is substantially isothermal throughout both zones.

The process can be conducted at any operable total pressure, ranging from subatmospheric to superatmospheric, provided that the vinyl aromatic product is produced. If the total reactor pressure is too high, the equilibrium position of the dehydrogenation process may be shifted backwards towards alkyl aromatic compound. On the other hand, an adequate steam pressure is needed to retard coking of the catalyst. Preferably, the process is conducted under a vacuum to maximize the yield of vinyl aromatic product. At the steam to oil mass ratios described hereinbefore, vacuum pressures are sufficient to regenerate the catalyst, at least in part. Preferably, the total pressure in the reactor is greater than about 1 psia (6.9 kPa). More preferably, the total pressure is greater than about 3 psia (20.7 kPa). Preferably, the total pressure is less than about 73 psia (503.3 kPa). More preferably, the total pressure is less than about 44 psia (303.4 kPa). Most preferably, the total pressure is subatmospheric, ranging between about 3 psia (20.7 kPa) and about 13 psia (90.6 kPa). The pressure throughout the freeboard, reaction, and regeneration zones may vary depending upon process factors, such as, the weight and buoyancy of the catalyst and frictional effects. Typically, the pressure is somewhat greater at the bottom of the reactor than at the top.

The space velocity of the dehydrogenation feedstream will depend upon the specific alkyl aromatic compound and catalyst used, the specific vinyl aromatic product formed, the reaction zone dimensions (for example, diameter and height), and the form and weight of the catalyst particles. It is desirable to remove the reactant and products quickly from the freeboard zone, so as to reduce thermal cracking and other undesirable side reactions. Additionally, the gas flow should be sufficient to induce fluidization of the catalyst bed. Generally, the space velocity of the dehydrogenation feedstream varies from the minimum velocity needed to achieve fluidization of the catalyst particles to a velocity just below the minimum velocity needed to achieve pneumatic transport of the catalyst particles. Fluidization occurs when the catalyst particles are disengaged, when the particles move in a fluid-like fashion, and when the bed pressure drop is essentially constant along the bed. Pneumatic transport occurs when a substantial quantity of catalyst particles are entrained in the gas flow and transported out of the reactor. Preferably, the space velocity of the dehydrogenation feedstream varies from the minimum bubbling velocity to the minimum turbulent flow velocity. Bubbling occurs when gas bubbles can be seen in the fluidized bed, but little back-mixing of gas and solids occurs. Turbulent flow occurs when there is both substantial bubbling and substantial back-mixing of gas and solids. More preferably, the flow is sufficiently high to induce back-mixing.

As a general guideline, the gas hourly space velocity (GHSV), calculated as the total flow of dehydrogenation feedstream, including alkyl aromatic compound and, optionally, steam, sweeping gas, and/or diluent flows, is greater than about 60 ml total feed per ml catalyst per hour (h⁻¹), measured at operating conditions. Preferably, the GHSV of the dehydrogenation stream is greater than about 120 h⁻¹, and more preferably, greater than 720 h⁻¹. Generally, the GHSV of the dehydrogenation stream is less than about 12,000 h⁻¹, preferably, less than about 3,600 h⁻¹, and more preferably, less than 1,800 h⁻¹, measured as the total flow at operating process conditions.

As a general guideline, the gas residence time in the reaction zone, calculated as the height of the reaction zone times the reaction zone voidage fraction divided by the superficial gas velocity total of the regeneration and reaction feedstreams, is greater than about 0.3 seconds (sec), measured at operating conditions. The “reaction zone voidage fraction” is the fraction of the reaction zone which is empty. The “superficial gas velocity” is the gas velocity through the empty reactor. Preferably, the gas residence time in the reaction zone is greater than about 1 seC, more preferably, greater than about 2 sec, measured at operating conditions. Generally, the gas residence time in the reaction zone is less than about 60 sec, preferably, less than about 30 sec, and more preferably, less than about 5 sec, measured at operating conditions.

The gas hourly space velocity of the regeneration feedstream through the regeneration zone can be broadly varied, provided that the catalyst is regenerated, at least in part, and provided that the catalyst particles in the regeneration zone are effectively fluidized. Again, the space velocity of the regeneration feedstream can vary from the minimum velocity needed to achieve fluidization of the catalyst particles to a velocity just below the minimum velocity needed to achieve pneumatic transport of the catalyst particles. Preferably, the space velocity of the regeneration feedstream varies from the minimum bubbling velocity to the minimum turbulent flow velocity. Typically, the gas hourly space velocity (GHSV), calculated as the total of the regeneration feedstream, is greater than about 60 ml total feed per ml catalyst per hour (h⁻¹). measured at operating conditions. Preferably, the GHSV of the regeneration stream is greater than about 120 h⁻¹, and more preferably, greater than about 360 h⁻¹. Generally, the gas hourly space velocity of the regeneration stream is less than about 12,000 h⁻¹, preferably, less than about 3,600 h⁻¹, and more preferably, less than about 720 h⁻¹, measured as the total flow at operating conditions.

In the regeneration zone, the gas residence time, calculated as the height of the regeneration zone times the regeneration zone voidage fraction divided by the superficial gas velocity of the total of the regeneration and reaction feedstreams, is greater than about 0.3 sec, measured at operating conditions. The “regeneration zone voidage fraction” is the fraction of the regeneration zone which is empty. Preferably, the gas residence time in the regeneration zone is greater than about 1 sec, and more preferably, greater than about 5 sec. Generally, the gas residence time in the regeneration zone is less than about 60 sec, preferably, less than about 30 sec, and more preferably, less than about 10 sec, measured at operating conditions.

When an alkyl aromatic compound and, optionally, steam are contacted with a dehydrogenation catalyst in the manner described hereinbefore, a vinyl aromatic compound is produced. Ethylbenzene is converted primarily to styrene. Ethyltoluene is converted to p-methylstyrene (p-vinyltoluene). t-Butylethylbenzene is converted to t-butylstyrene. Isopropylbenzene (cumene) is converted to α-methylstyrene, and diethylbenzene is converted to divinylbenzene. Hydrogen is also formed during dehydrogenation. Other products in smaller yields include benzene and toluene.

The conversion of the alkyl aromatic compound in the process of this invention can vary depending upon the specific feed composition, catalyst composition, process conditions, and fluidized bed conditions. For the purposes of this invention, “conversion” is defined as the mole percentage of alkyl aromatic compound which is converted to all products. In this process, the conversion of alkyl aromatic compound is typically greater than about 30 mole percent, preferably, greater than about 50 mole percent, and more preferably, greater than about 70 mole percent.

Likewise, the selectivity to products will vary depending upon the specific feed composition, catalyst composition, process conditions, and fluidized bed conditions. For the purposes of this invention, “selectivity” is defined as the mole percentage of converted alkyl aromatic compound which forms a specific product, preferably, the vinyl aromatic compound. In the process of this invention, the selectivity to vinvl aromatic compound, preferably styrene or a substituted derivative of styrene, is typically greater than about 60 mole percent, preferably, greater than about 75 mole percent, and more preferably, greater than about 90 mole percent.

The invention will be further clarified by a consideration of the following examples, which are intended to be purely illustrative of the use of the invention. Other embodiments of the invention will be apparent to those skilled in the art from a consideration of this specification or practice of the invention as disclosed herein. Selectivity measurements were corrected for the deviation from 100 percent in the organic material balance.

### EXAMPLE 1

A fluidized bed reactor [4.25 inches (10.63 cm) inner diameter; 20 inches (50 cm) height)] was constructed as shown in FIG. 1. The reactor comprised a single, vertical shell functionally divided into three zones: a catalyst regeneration zone (1) at the bottom of the reactor; a freeboard zone (3) at the top of the reactor; and a reaction zone (2) at the middle section between the regeneration and freeboard zones. A first inlet port (4) at the bottom of the reactor exited into a plenum area (10) in which a gas distributor (9) was located. This first inlet port was used to distribute a regeneration feedstream into the regeneration zone. A second inlet port (5), located in the freeboard zone, was used to introduce a dehydrogenation feedstream into the reaction zone (2). The second inlet port was connected to an inlet tube which terminated in a sparger array (6) in the reaction zone at a height of 3 inches (7.5 cm) above the bottom distributor plate (9). The sparger array, constructed as six rows of sintered metal tubing [Inconel, ¼ inch OD (6.3 mm OD)], was designed to provide a uniform pressure drop throughout the sparger. The exit ports in the sparger were positioned horizontally. An outlet port (7) was located in the freeboard zone for removing the product stream. Solids entrained in the product stream were collected in a cyclone (located below outlet port 7) and then recirculated to the reactor by means of a third inlet port (8) located in the regeneration zone. Effluent gases were collected downstream of the cyclone. The reactor was also equipped with a resistive (electrical) means for heating the reactor and two internal thermocouples (K type) for measuring the fluidized bed temperature in the reaction and regeneration zones.

The reactor was employed to dehydrogenate ethyl-benzene in the presence of a dehydrogenation catalyst to styrene, while simultaneously and continuously regenerating the dehydrogenation catalyst. A dehydrogenation catalyst (2370 g) with a mean particle diameter of 300 μm and comprising 28.7 percent iron oxide (Fe₂O₃), 14.3 percent cerium oxide (Ce₂O₃), 7.6 percent copper oxide (CuO), 31.6 percent potassium carbonate (K₂CO₃), 0.6 percent chromium oxide (Cr₂O₃), 9.5 percent zinc oxide (ZnO), and 7.6 percent cements, by weight, was loaded into the reactor. The reaction feedstream comprised a mixture of ethylbenzene and steam. The regeneration feedstream comprised steam. Gas products were analyzed using a Carle gas chromatograph equipped with a parallel array of five columns (2.7 percent Carbowax® 1540 on Porasil C; 3 percent Carbowax® 1540 on Porasil C; 27 percent Bis(EE)A on Chromosorb® PAW; Porapak® Q; and two 13X molecular sieve columns). Liquid products were analyzed using an HP 5890 gas chromatograph equipped with a J&W DB-5 column. Nitrogen was used as an internal standard for the gas analysis; while heptane was used as an internal standard for the liquid analysis. Sampling was done over the course of six hours and consisted of taking four or more samples every 30 minutes for the last few hours of operation. Ethylbenzene conversion and styrene selectivity results presented herein are averages of the four or more samples taken.

In the above-described reactor, water, at a feed rate of 4.3 cm3/min at room temperature, was heated to 600° C. and added via inlet port (4) to the plenum area (10) and through the distributor plate (9) into the regeneration zone (1) at the bottom of the reactor. Liquid ethylbenzene at a feed rate of 2.5 cm³/min and nitrogen gas at a feed rate of 1088 cm3/min at room temperature were mixed together, heated to 500° C. and added to the dehydrogenation reaction zone via inlet port (5) and the sparger array (6). The feed rates correspond to a total steam to oil mass ratio of 2/1 with a superficial velocity of 1.86 m/min in the regeneration zone and 237 m/min in the reaction zone. The gas residence time in the regeneration zone was 1.46 seconds; the gas residence time in the reaction zone was 0.67 seconds. The reactor temperature and pressure were maintained at 600° C. and 15.5 psia (106.9 kPa), respectively. Products obtained via exit port (7) were analyzed as noted above. Ethylbenzene conversion was 74.0 mole percent. Selectivity to styrene was 86.0 mole percent. Other products included benzene and toluene. The material balance accounted for 95 weight percent of the organic feed material.

### EXAMPLE 2

Using the reactor and catalyst of Example 1, water at a feed rate of 2.17 cm3/min was heated to 600° C. and added to the distributor plate in the regeneration zone. Liquid ethylbenzene at a feed rate of 2.52 cm³/min and liquid water at a feed rate of 2.17 cm3/min at room temperature were heated to 500° C. and added through the reaction zone to the sparger array. These feed rates correspond to a total steam to oil mass ratio of 2/1 with a superficial velocity of 156 m/min in the regeneration zone and 339.5 m/min in the reaction zone. The gas residence time in the regeneration zone was 2.91 seconds; the gas residence time in the reaction zone was 0.78 seconds. The reactor temperature and pressure were maintained at 600° C. and 15.5 psia (106.9 kPa), respectively. Ethylbenzene conversion was 85 mole percent. Selectivity to styrene was 69 mole percent. The material balance accounted for 96 weight percent of the organic feed material.

In Example 1, nitrogen was added as a sweeping gas to the ethylbenzene stream, but no steam was added to the ethylbenzene stream. In contrast in Example 2, no sweeping gas added to the ethylbenzene stream, and the steam stream was split between the dehydrogenation feed and the regeneration feed. When Example 2 was compared with Example 1, it was seen that the conversion of ethylbenzene was higher in Example 2 due to longer residence times in the bed, and the selectivity to styrene was lower due to increased free radical cracking in the freeboard region.

### EXAMPLE 3

Using the reactor and catalyst of Example 1, water at a feed rate of 4.3 cm3/min was heated to 600° C. and added to the distributor plate in the regeneration zone. Liquid ethylbenzene at a feed rate of 2.49 cm³/min at room temperature was heated to 500° C. and added through the reaction zone to the sparger array. These feed rates correspond to a total steam to oil mass ratio of 2/1 with a superficial velocity of 309 m/min in the regeneration zone and 417 m/min in the reaction zone. The gas residence time in the regeneration zone was 1.47 seconds; the gas residence time in the reaction zone was 0.63 seconds. The reactor temperature and pressure were maintained at 600° C. and 15.5 psia (106.9 kPa), respectively. Ethylbenzene conversion was 85 mole percent. Selectivity to styrene was 72 mole percent. The material balance accounted for 98 weight percent of the organic feed material.

The process conditions of Example 3 were closely similar to Example 2, with the following exception. In Example 2 one-half of the total steam was delivered to the regeneration zone, and one-half of the total steam was delivered to the reaction zone. In contrast, in Example 3 all of the steam was delivered to the regeneration zone. When Example 3 was compared with Example 2, it was seen that the conversion of ethylbenzene and the selectivity to styrene were comparable. Little difference was found which depended upon the location of introducing steam.

### EXAMPLE 4

Example 2 was repeated under closely similar process conditions, except for pressure which was held constant at 5 psia (34.5 kPa). The catalyst used in Example 4 had a chemical composition identical to the catalyst of the previous examples; however, the quantity of catalyst used was 1355 g, and the catalyst had a mean particle diameter of 220 μm. Process conditions were as follows: water feed rate to the regenerator zone at 2.9 cm3/min; liquid ethylbenzene and water feed rates to the reaction zone at 2.52 cm³/min and 1.45 cm³/min, respectively; steam to oil mass ratio at 2/1; superficial flow velocity in the regeneration zone at 123 cm/min; superficial flow velocity in the reaction zone at 200 cm/min; and temperature of 600° C. The ethylbenzene conversion was 49 mole percent. The styrene selectivity was 88 mole percent. A material balance accounted for 93 weight percent of the organic feed material.

A comparison of Examples 2 and 4 showed that significantly higher styrene selectivities can be obtained by operating the fluidized bed reactor under vacuum. The lower partial pressure of ethylbenzene feed somewhat lowers the overall conversion.

### EXAMPLE 5

Example 4 was repeated under closely similar process conditions, except that the reactor temperature was held constant at 590° C. rather than 600° C. Process conditions were as follows: water feed rate to the regenerator zone at 2.9 cm³/min; liquid ethylbenzene and water feed rates to the reaction zone at 2.52 cm³/min and 1.45 cm³/min, respectively; steam to oil mass ratio at 2/1; superficial flow velocity in the regeneration zone at 122 cm/min; superficial flow velocity in the reaction zone at 199 cm/min; and pressure of 5 psia (34.5 kPa). The ethylbenzene conversion was 50 mole percent. The styrene selectivity was 94 mole percent. A material balance accounted for 99 weight percent of the organic feed material. A comparison of Examples 4 and 5 showed that operating under vacuum with a temperature lower than 600° C. provides a further increase in styrene selectivity.

### EXAMPLE 6

Example 4 was repeated under closely similar process conditions, except that the reactor temperature was held constant at 580° C. rather than 600° C. Process conditions were as follows: water feed rate to the regenerator zone at 2.83 cm³/min; liquid ethylbenzene and water feed rates to the reaction zone at 2.52 cm³/min and 1.45 cm³/min, respectively; steam to oil mass ratio at 2/1; superficial flow velocity in the regeneration zone at 121 cm/min; superficial flow velocity in the reaction zone at 197 cm/min; and pressure of 5 psia (34.5 kPa). The ethylbenzene conversion was 44 mole percent. The styrene selectivity was 95 mole percent. A material balance accounted for 100 weight percent of the organic feed material. A comparison of Examples 4, 5, and 6 showed that operating under vacuum with a temperature lower than 600° C. provides a further increase in styrene selectivity.

### EXAMPLE 7

Example 4 was repeated under closely similar process conditions, except that the steam to oil ratio was 1/1 instead of 2/1. Other process conditions were as follows: water feed rate to the regeneration zone at 1.45 cm³/min; liquid ethylbenzene and water feed rates to the reaction zone at 2.52 cm³/min and 0.73 cm³/min, respectively; superficial flow velocity in the regeneration zone at 61.5 cm/min; superficial flow velocity in the reaction zone at 107.6 cm/min, pressure of 5 psia (34.5 kPa); and temperature of 600° C. The ethylbenzene conversion was 49 mole percent. The styrene selectivity was 89 mole percent. A material balance accounted for 98 weight percent of the organic feed material.

A comparison of Examples 4 and 5 with Example 7 showed that lowering the steam to oil ratio from 2/1 to 1/1 did not affect the ethylbenzene conversion and styrene selectivity.

### EXAMPLE 8

Example 4 was repeated under closely similar process conditions, except for catalyst particle size and steam to oil ratio. For Example 8, the catalyst (1570 g) had a mean particle diameter of 82 μm, and the steam to oil ratio was 0.5/1 Other process conditions were as follows: water feed rate to the regeneration zone at 0.8 cm³/min; liquid ethylbenzene and water feed rates to the reaction zone at 5.48 cm³/min and 0.54 cm³/min, respectively; superficial flow velocity in the regeneration zone at 33.52 cm/min; superficial flow velocity in the reaction zone at 74.8 cm/min, pressure of 5 psia (34.5 kPa); and temperature of 600° C. The ethylbenzene conversion was 54 mole percent. The styrene selectivity was 95 mole percent. A material balance accounted for 100 weight percent of the organic feed material.

Comparing Examples 4 and 7 with Example 8 showed that high styrene selectivity can be obtained with steam to oil ratios of 0.5/1. Furthermore, reducing the catalyst mean particle diameter from 220 μm to 82 μm resulted in an increase in the ethylbenzene conversion. This result is most likely due to better mass transfer since smaller catalyst particles tend to product smaller equilibrium bubble diameters in the fluidized bed reactor.

### EXAMPLE 9

A pulsed mode reactor was used to study the dehydrogenation of ethylbenzene to styrene as a function of time. In pulsed mode, the reactor was repeatedly cycled through a dehydrogenation step and thereafter a catalyst regeneration step. Experiments in pulsed mode reactors indicate what results can be expected in a fluidized bed reactor.

A dehydrogenation catalyst having particles sized between 1.18 mm and 1.70 mm and comprising 33.2 percent iron oxide (Fe₂O₃), 17.5 percent cerium oxide (Ce₂O₃), 7.8 percent copper oxide (CuO), 36.0 percent potassium carbonate (K₂CO₃), 0.6 percent chromium oxide (Cr₂O₃), and 4.7 percent cements, by weight, was loaded into a continuous flow, fixed-bed reactor [304 stainless steel, schedule 40, 1 inch (2.5 cm) OD×36 inches (90 cm) length)]. The catalyst bed occupied 7 inches (17.5 cm) of reactor length. The space above the bed was filled with ceramic berl saddles (¼ inch, 0.6 cm). Below the bed a metal spacer was situated. The temperature of the reaction was measured from a thermowell embedded in the catalyst bed. A dehydrogenation pulse was carried out by feeding ethylbenzene preheated to 550° C. over the catalyst for 2 min. The flow rate of the liquid ethylbenzene, measured at ambient temperature and pressure (taken as 23° C. and 1 atm), was 1.16 ml/min. Simultaneously, water preheated to 550° C. was fed over the catalyst during the same 2 min period. The flow rate of the water was adjusted to maintain a steam to oil weight ratio of 0.30/1. Total pressure was maintained at 5.0 psia. Thereafter, the ethylbenzene feedstream was stopped, and a regeneration pulse was conducted by feeding the water stream alone, preheated to 550° C., alone over the catalyst for 2 min under the same process conditions. The flow rate of the liquid water during the regeneration pulse was 1 ml/min, measured at 24° C. and 1 atm. Following regeneration, the dehydrogenation pulse was repeated, by reintroducing the ethylbenzene feedstream for 2 min as noted hereinbefore with the continuing water steam. After 2 min, the ethylbenzene feed was again stopped, while the steam stream was continued for a regeneration cycle of 2 min. The dehydrogenation-regeneration cycles were repeated for a total run time of 200 h. The product stream was continuously passed through a condenser, separated, and analyzed by conventional methods.

The results of the pulsed mode process are shown in FIG. 3, which plots the ethylbenzene conversion and styrene selectivity as a function of run time and at constant temperature (550° C.) and pressure (5.0 psia). Surprisingly, it was found that the ethylbenzene conversion increased slightly with time. Styrene selectivity remained constant at a value greater than 95 mole percent throughout the entire run. The results from the pulsed mode reactor indicated that the dehydrogenation catalyst could be cycled through dehydrogenation-regeneration steps in a fluidized bed reactor over long periods of time without significant deactivation.

### COMPARATIVE EXPERIMENT 1

The process of Example 9 was repeated in the same continuous flow, fixed bed reactor under similar process conditions, with the exception that the dehydrogenation was run in continuous mode rather than pulsed mode. Accordingly, there was only one dehydrogenation cycle and no catalyst regeneration cycle. Under these conditions, the catalyst was found to steadily deactivate with a concomitant decrease in ethylbenzene conversion. As the catalyst deactivated, the temperature of the process was increased to maintain a constant ethylbenzene conversion. At a steam to oil ratio of 0.3/1, the temperature had to be increased at a rate of 0.45° C. per min for conversion to be maintained. When Comparative Experiment 1 was compared with Experiment 9, it was found that the catalyst lifetime obtained in the pulsed mode reactor was significantly extended at constant temperature and pressure, whereas without regeneration the catalyst deactivated quickly and required increasing temperatures to maintain a constant conversion. The results from the pulsed mode reactor indicated that the dehydrogenation catalyst could be cycled through dehydrogenation-regeneration steps in a fluidized bed reactor over long periods of time without significant deactivation.

1. A process of dehydrogenating an alkyl aromatic compound over a dehydrogenation catalyst to form a vinyl aromatic compound, and regenerating the dehydrogenation catalyst in situ, the process comprising (a) fluidizing a dehydrogenation catalyst in a single shell fluidized bed reactor containing a reaction zone and a regeneration zone under fluidization conditions such that the catalyst is circulated within and between the two zones, (b) contacting a dehydrogenation feedstream comprising an alkyl aromatic compound, and optionally, steam with the dehydrogenation catalyst residing in the reaction zone under reaction conditions sufficient to prepare the corresponding vinyl aromatic compound; and (c) contacting a regeneration feedstream comprising steam with the dehydrogenation catalyst residing in the regeneration zone under regeneration conditions sufficient to regenerate, at least in part, the catalyst.

2. The process of claim 1 wherein the alkyl aromatic compound is a C₈-C₂₀ alkyl aromatic compound.

3. The process of claim 2 wherein the alkyl aromatic compound is ethylbenzene or a substituted ethylbenzene.

4. The process of claim 2 wherein the alkyl aromatic compound is selected from isopropylbenzene, diethylbenzene, and ethyltoluene.

5. The process of claim 1 wherein the fluidized bed reactor further comprises a freeboard zone, and a sweeping gas is added to the freeboard zone.

6. The process of claim 1 wherein the total steam to alkyl aromatic compound weight ratio is greater than about 0.2/1 and less than about 5.0/1.

7. The process of claim 1 wherein the total steam to alkyl aromatic compound weight ratio is greater than about 0.2/1 and less than about 1.2/1.

8. The process of claim 1 wherein a diluent gas is fed with the dehydrogenation feedstream, or fed with the regeneration feedstream, or fed with both streams.

9. The process of claim 8 wherein the diluent gas is selected from nitrogen, argon, helium, carbon dioxide, steam, and mixtures thereof.

10. The process of claim 8 wherein the diluent comprises from greater than about 10 volume percent to less than about 90 volume percent of the dehydrogenation or regeneration feedstream, or both streams independently.

11. The process of claim 1 wherein the dehydrogenation feedstream is preheated to a temperature greater than about 150° C. and less than about 600° C.

12. The process of claim 1 wherein the regeneration feedstream is preheated to a temperature greater than about 200° C. and less than about 650° C.

13. The process of claim 1 wherein the temperature in the reaction and/or regeneration zones is greater than about 550° C. and less than about 650° C.

14. The process of claim 1 wherein the total pressure in the reactor is greater than about 1 psia (6.9 kPa) and less than about 73 psia (503.3 kPa).

15. The process of claim 1 wherein the process is conducted at a gas hourly space velocity, calculated as the total flow of the dehydrogenation feedstream, of greater than about 60 h⁻¹ and less than about 12,000 h⁻¹, measured at operating process conditions.

16. The process of claim 1 wherein the process is conducted at a residence time of total gas flow in the reaction zone of greater than about 0.3 seconds and less than about 60 seconds, measured at operating process conditions.

17. The process of claim 1 wherein the process is conducted at a gas hourly space velocity, calculated as the total flow of the regeneration feedstream, of greater than about 60 h⁻¹ and less than about 12,000 h⁻¹, measured at operating process conditions.

18. The process of claim 1 wherein the process is conducted at a gas residence time in the regeneration zone of greater than about 0.3 seconds and less than about 60 seconds, measured at operating process conditions.

19. The process of claim 1 wherein the dehydrogenation catalyst comprises iron oxide.

20. The process of claim 19 wherein the dehydrogenation catalyst further comprises at least one or more compounds selected from the compounds of alkali metals, alkaline earth metals, chromium, gallium, cerium, zinc, and copper.

21. The process of claim 19 wherein the dehydrogenation catalyst comprises (a) at least one iron oxide, (b) at least one carbonate, bicarbonate, oxide or hydroxide of potassium and/or cesium, (c) an oxide, carbonate, nitrate or hydroxide of cerium, (d) optionally, a hydroxide, carbonate, bicarbonate, acetate, oxalate, nitrate, or sulfate of sodium, (e) optionally, a carbonate, sulfate, or hydroxide of calcium, (f) optionally, one or more compounds of zinc, chromium, and copper, and (g) optionally, a cement.

22. The process of claim 1 wherein the conversion of alkyl aromatic compound is greater than about 30 mole percent.

23. The process of claim 1 wherein the selectivity to vinyl aromatic compound is greater than about 60 mole percent.

24. The process of claim 1 wherein the vinyl aromatic compound is styrene or a substituted derivative of styrene.

25. The process of claim 24 wherein the substituted styrene is selected from divinylbenzene, α-methylstyrene, and vinyltoluene.

26. The process of claim 1 wherein the average particle size of the dehydrogenation catalyst is greater than about 20 microns and less than about 1,000 microns.
The process of claim 1 wherein the fluidized bed reactor comprises a single vertical shell enclosing a freeboard zone, a reaction zone, and a regeneration zone; an inlet means for introducing the regeneration feedstream into the regeneration zone and an inlet means for introducing a reactant feedstream into the reaction zone, one of said inlet means into the reaction or regeneration zones being capable of separating the two zones while allowing for the circulation of catalyst particles between the two zones; and further comprising an outlet means for an effluent stream; and optionally, an inlet means for returning catalyst entrained with the effluent stream to the reactor; and optionally, an inlet and outlet means for conveying catalyst into and out of the reactor.

28. The process of claim 27 wherein the means for separating the reactant and regeneration zones comprises a sparger array or distributor.

29. A process of dehydrogenating ethylbenzene or a substituted ethylbenzene over a dehydrogenation catalyst to form styrene or a substituted styrene, and regenerating the dehydrogenation catalyst in situ, the process comprising (a) fluidizing a dehydrogenation catalyst in a single shell fluidized bed reactor containing a reaction zone and a regeneration zone under fluidization conditions such that the catalyst is circulated within and between the two zones, (b) contacting ethylbenzene or a substituted ethylbenzene, and optionally steam, and optionally a diluent gas, with the dehydrogenation catalyst residing in the reaction zone, the catalyst comprising iron oxide, and the contacting being conducted at a steam to ethylbenzene weight ratio greater than about 0.2/1 and less than about 3.0/1, a temperature greater than about 570° C. and less than about 610° C., and a total reactor pressure greater than about 3 psia (41 kPa) and less than about 44 psia (302 kPa; and (c) contacting the dehydrogenation catalyst residing in the regeneration zone with a regeneration feedstream comprising steam, and optionally, a diluent at a temperature greater than about 570° C. and less than about 610° C., so as to regenerate, at least in part, the catalyst.

30. A fluidized bed reactor for catalyzed organic processes with in situ catalyst regeneration comprising, a single vertical shell enclosing a freeboard zone, a reaction zone, and a regeneration zone; an inlet means for introducing a regeneration feedstream into the regeneration zone and an inlet means for introducing a reactant feedstream into the reaction zone, one of said inlet means being capable of separating the reaction and regeneration zones while allowing for the circulation of catalyst particles between the two zones; and further comprising an outlet means for an effluent stream; and optionally, an inlet means for returning catalyst entrained with the effluent stream to the reactor.

31. The fluidized bed reactor of claim 30 wherein the means for introducing the reactant feedstream comprises a sparger array or distributor.

32. The fluidized bed reactor of claim 30 wherein the means for introducing the regeneration feedstream comprises a sparger array or distributor.

33. The fluidized bed reactor of claim 30 wherein the means for separating the reaction and regeneration zones is selected from a sparger array or a distributor.

34. The fluidized bed reactor of claim 30 further comprising a means for enhancing solids circulation.

35. The fluidized bed reactor of claim 34 wherein the means for enhancing solids circulation comprises a draft tube, optionally, containing internal baffles.

36. The fluidized bed reactor of claim 34 wherein the means for enhancing solids circulation comprises a draft tube made of heating or cooling elements.

37. The fluidized bed reactor of claim 34 further comprising an inlet means and an outlet means for conveying catalyst into and out of the reactor.

38. The fluidized bed reactor of claim 34 further comprising at least one means for measuring the temperature of the fluidized bed and, optionally, a heating means.
